(19) United States
(12) Patent Application Publication
(10) Pub. No.: US 2013/0177597 A1
(43) Pub. Date: Jul. 11, 2013

(54) Mango seed herbal medication

(71) Applicants: Jolly Cherian, Coral Springs, FL (US); Ninan Cherian, Coral Springs, FL (US); Diana Cherian, Coral Springs, FL (US)

(72) Inventors: Jolly Cherian, Coral Springs, FL (US); Ninan Cherian, Coral Springs, FL (US); Diana Cherian, Coral Springs, FL (US)

(21) Appl. No.: 13/374,656

(22) Filed: Jan. 6, 2012

(51) Int. Cl. A61K 36/22; A61K 36/288; A61P 7/10; A61P 37/04; A61P 13/12; A61P 29/00; A61K 36/36; A61P 39/02

(52) U.S. Cl. 424/278.1; 424/764; 424/776

(57) ABSTRACT

An herbal composition for treatment of kidney nephrotic syndrome formed of a mixture of the herbs mango seed powder, Mangifera Indicia; dandelion, Taraxacum officinale; fennel seed, Foeniculum Vulgare; spreading hogweed, Boerhavia diffusa; and nettle plant, Urtica dioica.

### BACKGROUND OF THE INVENTION

The present invention relates to an herbal medication mixture and particularly to an herbal mixture made with mango seed powder.

Mango fruit originated in tropical locations and Asian countries and now is one of the most popular fruits of the world. The mango has a rich, aromatic flavor and has been used for a wide variety of culinary applications. In addition to taste, the mango has been recognized as having many restorative and medical properties. It is a powerful source of nutrition containing essential vitamins, minerals, such as potassium, calcium and iron, enzymes and anti-oxidants necessary for good physical health. In addition, the ripe fruit is used as a laxative, diuretic anti-hemorrhagic, ophthalmic, astringent, anti-diarrhea, and restorative tonic. Mango has been used for clearing of gall and kidney stones and as a natural remedy for diabetes. Mango seed powder is particularly useful to eliminate diarrhea and dysentery.

### SUMMARY OF THE INVENTION

The present invention relates to an herbal composition made with interactive ingredients for treatment of kidney disorders such as nephrotic syndrome, comprising mango seed powder, Mangifera Indica and at least one other active ingredient from a group including dandelion, Taraxacum officinale, fennel seed, Foeniculum Vulgare, spreading hogweed, Boerhavia diffusa, and nettle plant, Urtica dioica. The mango seed powder removes toxins from the body through the kidneys. The dandelion also eliminates waste through the kidneys. The fennel seed is an anti-inflammatory ingredient and also acts to strengthen the immune system. The spreading hogweed is a diuretic and aids in increased kidney flow to eliminate toxins without additional strain on the kidneys. The nettle plant is rich in iron and helps strengthen the immune system while alleviating anemia and fatigue.

In a preferred embodiment, of the composition, a combination of all the ingredients is mixed together to form the herbal mixture. In this embodiment the mixture is prepared as follows. The shell of the mango seed is opened and the kidney shaped white interior flesh is removed from the hard outer shell. The interior flesh is dehydrated and made into a powder. The dandelion leaf is dehydrated. Dry fennel seed is powdered. The flower of the spreading hogweed is dried and then powdered. The nettle plant leaf is also dried and powered.

The above ingredients are mixed in the following proportions. The main ingredient is mango seed powder and it makes up to 90 percent of the herbal mixture. The remaining ingredients, that is, the dandelion, fennel seed, spreading hogweed flower and the nettle plant leaf is added in approximately equal amounts from as little as 1 or 2 percent up to as much as 10 percent.

The most preferred mixture uses 80 percent mango seed powder, 5 percent dandelion leaf, 5 percent fennel seed powder, 5 percent spreading hogweed flower powder and 5 percent nettle plant leaf.

In another embodiment, the mango seed powder is combined with at least one additional herbal ingredient selected from the group consisting of dandelion, fennel seed, spreading hogweed flower and nettle plant leaf.

An object of the present invention is the provision of an herbal composition for the treatment of nephrotic kidney disorder.

### DETAILED DESCRIPTION OF THE INVENTION

The present invention provides herbal compositions useful in the treatment of kidney disorders and in particular in the treatment of nephrotic syndrome and methods of making the compositions. Nephrotic syndrome is a disorder of the kidneys causing them to leak large amounts of protein from the blood into the urine. Whereas many herbal remedies have been used for kidney disorders, the present herbal composition is extremely effective to reverse the symptoms of nephrotic syndrome.

The primary embodiments of the herbal composition comprise a composition of mango seed powder, Mangifera Indica, dandelion, Taraxacum officinale, fennel seed, Foeniculum vulgare, spreading hogweed, Boerhavia diffusa, and nettle plant, Urtica dioica.

Mango seed, Mangifera Indica, powder, finds a variety of medical uses including treatment of diarrhea and dysentery, hemorrhage, hemorrhoids, disorders of female reproductive organs and gum and dental disorders. The fruit is used in a wide variety of medical disorders. Mango leaves helps eliminate gall and kidney stones. Mango seed powder is particularly useful to remove toxins from the body through the kidneys.

Dandelion, Taraxacum officinale, is a rich source of vitamins A, B complex and D and minerals including iron, potassium and zinc. Dandelion is used as a food in salads and as a drink for tea and wine. The roots and leaves have been used to treat liver problems, kidney disease, swelling, skin problems, heartburn and upset stomach as well as for fever, boils, diabetes and diarrhea. Other uses include use as a diuretic to get rid of excess fluid.

Fennel seed comes from fennel, Foeniculum vulgare, a perennial umbelliferous herb that is highly aromatic and flavorful and used for culinary and medical applications including snakebite, obesity and eye problems. In addition, fennel seed is antiseptic and secretolytic, that is encouraging secretion of gastric juices and saliva. It also can be used to treat stomach problems and act as a laxative. Fennel seed powder has essentially the same effect as the seed itself.

Spreading hogweed, Boerhavia diffusa, Panarnava, is an herbal plant that has anti-inflammatory, analgesic, anti-convulsion, diuretic laxative and expectorant properties. It has been used for obesity, anemia, appetite loss, skin infections and as a blood purifier. As a diuretic it aids the kidneys to eliminate toxins without strain on the kidneys.

Nettle plant, Urtica dioica, is well known in medicine, folk remedies and cooking applications. The nettle plant helps strengthen the immune system. It has been used for respiratory disease, blood problems skin disease, as well as kidney and urinary tract disease.

The main ingredient of the subject herbal composition is mango seed powder and it makes up to 90 percent or more of the herbal mixture. The remaining ingredients, that is, the dandelion, fennel seed, spreading hogweed flower and the nettle plant leaf are added in approximately equal amounts from as little as 1 or 2 percent up to as much as 10 percent.

The ingredients forming the herbal composition are preferably made into a powder and combined. Initially, the shell of the mango seed is opened and the kidney shaped white interior flesh is removed from the hard outer shell. The interior flesh is dehydrated and made into a powder. The dandelion leaf is dehydrated and powdered. Dry fennel seed is powdered. The flower of the spreading hogweed is dried and then powdered. The nettle plant leaf is also dried and powered.

A preferred embodiment is formed of a mixture of 80 percent mango seed powder, 5 percent dandelion leaf, 5 percent fennel seed powder, 5 percent spreading hogweed flower powder and 5 percent nettle plant leaf.

The main ingredient, mango seed powder, removes toxins from the body through the kidneys. The dandelion also eliminates waste through the kidneys. The spreading hogweed is a diuretic and aids in increased kidney flow to eliminate toxins without additional strain on the kidneys. The fennel seed is an anti-inflammatory ingredient and also acts to strengthen the immune system. The nettle plant is rich in iron and also helps strengthen the immune system while alleviating anemia and fatigue.

In accordance with the present invention, a particular embodiment of the herbal composition uses mango seed powder and an herbal ingredient such as dandelion or spreading hogweed for removing toxins through the kidneys and an ingredient such as fennel seed or nettle plant for strengthening the body's immune system.

The herbal composition may be administrated in a variety of different ways well known in the healing arts. Vehicles for delivering dosages may be tablets, capsules, powders, patches, liquid solutions and suspensions. The herbal composition preferably is administrated orally, but other deliveries are contemplated such as transdermal, intrapulmonary, rectal and so forth, as recognized in the healing arts.

It will also be appreciated that the herbal composition of the present invention may be combined with or contain one or more conventional pharmaceutical acceptable excipients, adjuvants, solvents or carriers and may, also include flavorings, colorings, coatings and similar well known materials.

Whereas specific parts of each herbal ingredient are contemplated, such as the seeds, flowers, leaves and so forth, it will be appreciated that the present combination of herbal ingredients contemplates using any and all parts of the herb including the flowers, fruit, leaves, stems and roots in various combinations.

1. An herbal composition comprising the herbs mango seed, Mangifera Indicia; dandelion, Taraxacum officinale; fennel seed, Foeniculum Vulgare; spreading hogweed, Boerhavia diffusa; and nettle plant, Urtica genus.

2. The herbal composition of claim 1 further defined as a mixture of up to 90 percent mango seed and ten percent of dandelion, fennel seed, spreading hogweed and nettle plant.

3. The herbal composition of claim 1 further defined as a mixture of eighty percent mango seed; five percent of dandelion; five percent of fennel seed; five percent spreading hogweed; and five percent nettle plant.

4. The herbal composition of claim 1 further defined as consisting of eighty percent mango seed; five percent dandelion; five percent fennel seed; five percent spreading hogweed; and five percent nettle plant.

5. The herbal composition of claim 1 wherein said mango seed is characterized as being powdered.

6. The herbal composition of claim 1 wherein said dandelion is characterized as dehydrated dandelion leaf.

7. The herbal composition of claim 1 wherein said fennel seed is characterized as powdered fennel seed.

8. The herbal composition of claim 1 wherein said spreading hogweed is characterized as dried and powdered spreading hogweed flowers.

9. The herbal composition of claim 1 wherein said nettle plant is characterized as dried and powdered stinging nettle leaf.

10. An herbal composition comprising mango seed powder and at least one herb from the group consisting of dandelion; fennel seed; spreading hogweed; and nettle plant.

11. The herbal composition of claim 10 further defined as at least one herb for removing toxins from the group consisting of dandelion and spreading hogweed and at least one herb for strengthening the immune system from the group consisting of fennel seed and nettle plant.

12. The herbal composition of claim 10 formed with suitable binders to form a capsule or tablet dosage form.
